(19) United States
(12) Patent Application Publication
(10) Pub. No.: US 2017/0281512 A1
(43) Pub. Date: Oct. 5, 2017

(54) COSMETIC POWDER TREATED WITH POLYSACCHARIDE AND METHODS OF MAKING THE SAME

(71) Applicant: Miyoshi America Inc., Dayville, CT (US)

(72) Inventor: Shoji Takekawa, Shrewsbury, MA (US)

(21) Appl. No.: 15/084,071

(22) Filed: Mar. 29, 2016

(51) Int. Cl. A61K 8/73; A61K 8/29; A61K 8/26; A61Q 1/02; A61K 8/19; A61Q 19/00; A61Q 19/10; A61K 8/02; A61K 8/25

(52) U.S. Cl. CPC A61K 8/73; A61K 8/022; A61K 8/29; A61K 8/26; A61K 8/25; A61K 8/19; A61K 8/733; A61K 8/731; A61K 8/732; A61Q 19/00; A61Q 19/10; A61Q 1/02; A61K 2800/614; A61K 2800/805; A61K 2800/52

(57) ABSTRACT

The present disclosure relates generally to cosmetic powders in which the powders are surface modified with at least one polysaccharide, or salt forms thereof. The powders may be used in cosmetic compositions such as a powder foundation, liquid foundation, point makeup, lip, mascara, eyeliner, skin care products which are skin cream, hair care products which are shampoo, conditioner, treatment and hair styling products, hair color products, cleansing products which are a body soap, hand soap and facial cleanser.

### FIELD OF THE INVENTION

The present disclosure relates generally to cosmetic powder materials having a surface treated with at least one polysaccharide, cosmetic formulations containing the surface treated powder, and methods of making a cosmetic powder surface treated with at least one polysaccharide. The cosmetic powders are readily dispersible in water, have improved stability, and provide an easy to wash property when present in cosmetic compositions.

### BACKGROUND

The information provided below is not admitted to be prior art to the present invention, but is provided solely to assist in a more complete understanding of the embodiments.

A significant amount of various powders are conventionally used for making makeup, skincare products, toiletries, and other products marketed and distributed by the personal care industry. Powders dispersed in various product forms such as water base solution, water gel, w/o and o/w formulas, may suffer from poor dispersibility and product stability, which can result in the formation of aggregates, agglomerates and flocculation. These results can be due to the nature of powder's physical properties, including particle size, surface activity, charge, polarity and specific gravity, to name a few.

Untreated powder agglomerates easily due to several surface properties (including surface charge, surface polarity etc.). In order to solve this problem and to thereby improve dispersibility and stability of powders, surface treatments with various treating agents have been proposed. Agents and methods for surface treating powders vary depending on the aim of the treatment. A treating agent may be selected in view of properties of the surface to be treated and its interaction with a dispersion medium. Known methods include, for instance, lipophilization with oils or metal soaps, hydrophilization treatment with surfactants or silica, and hydrophobization with silicone oils.

There are many types of lipophilization treatments, but there are relatively few known conventional hydrophilization treatments, such as silica treatments. Current hydrophilization treatments that are known are not entirely satisfactory. For example, in a composition formulated with a hydrophilic treated powder, the treating agent sometimes separates from the powder to cause agglomeration of the powder. This may result in mottles and color differences between the desired coating color and the resultant applied color. In addition, re-dispersibility sometimes worsens, which is inconvenient in its use and may cause product stability issues. Further, some of the known surfactants used in aqueous system cause skin irritation, which is problematic in personal products.

In recent years, powders have been developed to provide long lasting cosmetics with a smoother consistency. In obtaining these desirable traits, the focus has largely been on the hydrophobic properties of the surface treatments on powders and pigments, and improvements in the dispersibility of surface treated powders into an oil phase. However, when powders are used in cosmetic systems, such as foundations, lip sticks, lotions, or creams, the powders typically have to be dispersed in an aqueous phase, due to the hydrophilic nature of most cosmetic powders. To disperse non-hydrophobic powders in an aqueous phase, multiple emulsifiers are often used. Without these emulsifiers, dispersions in water-based systems often become problematic. The use of emulsifiers can be disadvantageous, however, with respect to producing a sticky, heavy feeling to the composition.

The affinity of a powder is dependent on surface characteristics of the powder, such as particle size, particularly nano-sized and micro-sized powders and the aspect ratio of powder. The high affinity of residual powders on the substrates that are commonly used in skin and hair consumer products often requires additional wash steps or specialized cleansing products to remove them completely. Furthermore, personal care products containing powders such as color pigments often make bathroom surfaces dirty, thus requiring cleaning. Pearlescent pigments are often used in skin care and hair care products to attract consumers for aesthetic purposes. However, the manufacturing process for skin and hair care products that use pearlescent pigments may cause a cleaning issue because pearlescent pigment residue is very difficult to remove completely due to its high luminance property and thus it requires additional cleaning steps to avoid cross contamination.

It also is known that direct contact of inorganic and organic cosmetic powders with the skin may lead to the absorption of water on the skin surface, thus altering the natural hydrophilic and lipophilic balance, which may cause localized dehydration effects and consequently an unpleasant feeling by those using these products. In addition, the lack of homogeneity of the powders used, having different physical features from one another, may ultimately generate clearly perceptible defects. Cosmetic powders therefore are typically treated to modify the surface of the powder to provide improved dispersibility, homogeneity and stability and to reduce the deleterious effects caused by direct contact with the skin.

There are proposed a variety of surface-treating methods. In one method, a silicone oil (for instance, methyl polysiloxane, methyl hydrogen polysiloxane or alkyl silane with the number of carbon atoms of an alkyl portion being not more than 10) is dissolved into a solvent as a surface-treating agent, which then is added and mixed into a powder, and the surface treatment is baked onto the powder by heating after the drying process. In another method, while a powder and octyl triethoxy silane or the like are being dispersed into an organic solvent by using a media grinder, the surface of the powder is treated with an organic silicon compound such as octyl triethoxy silane (JP-A 08-104606). Another method involves stirring and mixing with a Henschel mixer N-octyl trimethoxy silane or N-octyl triethoxy silane as an alkyl silane compound, and a reaction is completed with the powder under heating, and the resultant treated powder is pulverized by a hammer mill (JP-A 2001-181136). In another method, a silicone compound such as methyl hydrogen polysiloxane or the like is emulsified by dispersing it in water, and surfaces of powder particles are coated by mixing the emulsion to the powder (JP-A 09-268271).

JP-B 06-59397 discloses a jet method in which after a metal soap, an organic silicon compound in which a reactive group such as a hydrogen group or the like is bonded to a silicon atom, and a powder are mixed, the mixture is pulverized by a miller using an ejecting stream simultaneously with the surface treatment. JP-A 2002-80748 discloses a method in which in order to improve dispersability of a powder, coating is effected with surface treating agents for an A layer and a layer B by a jet method. Another method involves mixing a silica compound in water, ethanol and aqueous ammonia, and therein dispersing titania powder to prepare a pre-mix 1. Separately, tetraethoxysilane, water and ethanol were mixed to prepare pre-mix 2. Pre-mix 2 was added to pre-mix 1 under stirring with a magnetic stirrer, at a constant rate over 2 hours. The mixture obtained was aged for 12 hours. The coating formation and aging were performed at 25° C. Thereafter, the solution was filtered by suction and the filtrate was dried with hot air at 50° C. for 12 hours to obtain silica-coated powder. This process is disclosed in U.S. Pat. No. 6,534,044, the disclosure of which is incorporated by reference herein in its entirety.

U.S. Pat. No. 5,496,544, the disclosure of which is incorporated by reference herein in its entirety, discloses a skin cosmetic composition consisting of an anhydrous powder comprising a solid powder phase mixed with a fat-based binder which contains a silicone mixture comprising at least one silicone oil, at least one silicone wax, at least one silicone resin, and optionally at least on silicone rubber and optionally at least one phenyl dimethicone. However, in U.S. Pat. No. 5,496,544, the anhydrous powder undergoes a physical treatment by said fat-based binder. Therefore, in the cosmetic composition from U.S. Pat. No. 5,496,544, the absence of a covalent chemical bond between the powder phase and fat-based binder has the drawback of an easy extraction of the latter from the powder phase. Also, in the cosmetic composition from U.S. Pat. No. 5,496,544, the powder phase coating consists of complex mixtures of silicones which confer a different kind of sensorial effects on the skin itself

EP 1 116 753 describes a powder treated with reactive silicone comprising a powder surface-coated with a silicone compound, in which the amount of hydrogen generated from Si—H groups left on the surface of the silicone-treated powder is not greater than 0.2 ml/g of the treated powder and a contact angle between the water and the treated powder is at least 100°. However, the direct reaction between methyl hydrogen polysiloxane containing reactive Si—H bonds and the powder surface described in EP 1 116 753 fails to reach completion and it has the disadvantage to release some H₂ over time, which is the cause of several drawbacks for the obtained cosmetic powder. Indeed, on the one hand the generation of H₂ may cause the containers carrying the powder to swell and deteriorate, on the other hand the powder itself may harden and break.

It would be desirable to formulate cosmetic compositions with powders that have been surface treated to improve the powder's dispersibility and stability in the composition. Other objects and advantages will become apparent from the following disclosure. While certain drawbacks and disadvantages have been described with respect to the state of the art, the embodiments described herein are not to be construed to exclude some or all of the features described above. Indeed, aspects of the embodiments may include features known in the art, without suffering from their previously known adverse effects.

### SUMMARY OF INVENTION

The embodiments described herein relate to a surface treated cosmetic powder in which the powder has been surface treated with at least one polysaccharide, and salt forms thereof. In accordance with an embodiment, there is provided at least one cosmetic powder in which the surface of the at least one cosmetic powder is chemically modified with at least one polysaccharide or mixtures thereof, and salt forms thereof, wherein the polysaccharide is chemically immobilized on the surface of the at least one powder.

Another embodiment relates to a method for making a surface-modified cosmetic powder that includes: (a) preparing an aqueous solution of polysaccharide; (b) adding to the aqueous solution at least one cosmetic powder with agitation to uniformly disperse the powder in the aqueous mixture; and (c) adding a metal-containing salt to neutralize the aqueous mixture and chemically immobilize the polysaccharide on the surface of the at least one cosmetic powder. The method also may include drying after neutralization.

According to other embodiments, there is provided a cosmetic formulation that includes: (a) at least one cosmetic powder in which the surface of at the least one cosmetic powder is chemically modified with at least one polysaccharide, and salt forms thereof, wherein the polysaccharide is chemically immobilized on the surface of at least one powder; and (b) a cosmetically acceptable carrier.

Still other aspects and advantages of the embodiments will become readily apparent to those having ordinary skill in the art from the following detailed description, wherein particularly preferred embodiments are shown and described, simply by way of illustration. As will be realized the preferred embodiments include other and different embodiments, and its several details are capable of modifications in various obvious respects. Accordingly, the description is to be regarded as illustrative in nature and not as restrictive.

### BRIEF DESCRIPTION OF DRAWINGS

The embodiments can be understood from the following detailed description when read in connection with the accompanying drawings. Included in the drawings are the following figures:

FIG. 1 is a comparison of the dispersibility of untreated titanium dioxide over time (FIG. 1A), with the dispersibility of titanium dioxide treated in accordance with the embodiments (FIG. 1B), and with the dispersibility of titanium dioxide treated with silica (FIG. 1C). Each line (except the red line) shows the settling of powder every 10 minute. If powder settles down, the line moves from Right-Top to Left-Bottom. The red line shows 24 hours after initial sample measurement.

FIG. 2 illustrates a comparison of the washability of untreated powder and powder treated with varying amounts of polysaccharide in accordance with the embodiments.

It is to be noted, however, that the appended drawings illustrate only typical embodiments of this invention and are therefore not to be considered limiting in its scope, for the invention may admit to other equally effective embodiments.

### DETAILED DESCRIPTION OF A PREFERRED EMBODIMENT

Reference is made to the figures to illustrate selected embodiments and preferred modes of carrying out the invention. It is to be understood that the embodiments are not hereby limited to those aspects depicted in the figures.

The following definitions and non-limiting guidelines are provided to assist in better understanding the detailed description of herein. The headings (such as “Background” and “Brief Summary,”) and sub-headings used herein are intended only for general organization of topics within the disclosure of the embodiments, and are not intended to be limiting. For example, subject matter disclosed in the “Background” may include aspects of technology within the scope of the embodiments, and may not constitute a recitation of prior art. Subject matter disclosed in the “Brief Summary” is not an exhaustive or complete disclosure of the entire scope of the embodiments. Classification or discussion of a material within a section of the specification as having a particular utility (e.g., as being an “active” or a “carrier” ingredient) is made for convenience, and no inference should be drawn that the material must necessarily or solely function in accordance with its classification herein when it is used in any given composition.

The citation of references herein does not constitute an admission that those references are prior art or have any relevance to the patentability of the embodiments disclosed herein. Any discussion of the content of references cited in the Background is intended merely to provide a general summary of assertions made by the authors of the references, and does not constitute an admission as to the accuracy of the content of such references.

The description and specific examples, while indicating embodiments, are intended for purposes of illustration only and are not intended to be limiting. Moreover, recitation of multiple embodiments having stated features is not intended to exclude other embodiments having additional features, or other embodiments incorporating different combinations the stated of features. Examples are provided for illustrative purposes of how to make and use the compositions and methods described herein, unless explicitly stated otherwise, are not intended to be a representation that given embodiments have, or have not, been made or tested.

As used herein, the words “preferred” and “preferably” refer to embodiments that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope thereof. In addition, the compositions and the methods may comprise, consist essentially of, or consist of the elements described therein.

As used throughout, ranges are used as a short-hand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

Throughout this description, the use of the term “about” or “approximately” is intended to denote an approximation of the number, which includes the number modified by the term, and a reasonable deviation from that term, including standard measurement errors. Unless otherwise specified, all percentages and amounts expressed herein and elsewhere in the specification should be understood to refer to percentages by weight. The amounts provided are based on the active weight of the material. The recitation of a specific value herein is intended to denote that value, plus or minus a degree of variability to account for errors in measurements. For example, an amount of 10% or about 10% may include 9.5% or 10.5%, given the degree of error in measurement that will be appreciated and understood by those having ordinary skill in the art.

As used herein, the term “cosmetic composition” means a composition that is intended to be applied onto the consumer's skin, particularly onto the facial skin or onto the body skin area or onto hair, so as to regulate the condition of the skin and/or to improve the appearance of the skin and hair. The term “powder” denotes any material having a particle size within the range of from about 0.01 micrometer to 100 micrometers used for cosmetics. The term “average primary particle size” of powder treated with polysaccharide denotes the equivalent volume mean primary particle size of the elementary powder treated with polysaccharide. The average primary particle size is measured on the powder treated with polysaccharide, before being treated.

Throughout this description, the term “foundation” means a cosmetic composition that is intended to be applied onto the consumer's skin, particularly, onto the facial skin, body skin and hair so as to provide coverage and/or to mask skin irregularities and/or skin imperfections and/or skin tonal variations. The term “chalkiness” means the white hue which is observed onto skin after applying onto skin, particularly darker skin. The term “pastiness” means the white hue that may be observed on the skin after applying onto skin, particularly lighter skin.

All percentages, ratios and proportions herein are by weight, unless otherwise specified. All such weights as they pertain to listed ingredients are based on the active level, unless otherwise specified.

Embodiments described herein include one or more cosmetic powders having been treated with at least one polysaccharide in which the surface of the powder is modified by chemically immobilizing the at least one polysaccharide onto the surface of the powder. Cosmetic powders typically include substrates, pigments, and extenders. Substrates and pigments typically comprise or consist of a material compatible or acceptable for cosmetic and makeup products, personal care products and pharmaceutical products. Substrates and pigments are typically in the form of a powder, which is a solid, dry material consisting of small, flowable particles. Particular classes of powder materials are inorganic and organic particles, beads, crystals, clays, metals, metal oxide powders, plastics and fillers for plastic suitable for cosmetic use.

The at least one polysaccharide surface treatment agent can be chemically immobilized or adsorbed onto the surface cosmetic powder. Chemical linkage or immobilization of the polysaccharide surface-treatment agent, or salt thereof, to a cosmetic powder differs from adsorption in that surface treated material has a more uniformly chemically bound reaction product. Chemical linkage or immobilization tends to reduce movement and/or rearrangement of any material linked or attached onto the surface of the modified powder material. For example, a polysaccharide surface treatment agent, or salt thereof, that is linked or attached to the surface of a cosmetic powder will have less mobility than a treatment agent that is attached or linked to the surface of a powder by virtue of adsorption.

In order to facilitate or enhance immobilization of surface-treatment agents to the cosmetic powder, a reaction may be created by a water soluble compound having a lipophilic or hydrophilic moiety being absorbed onto the surface of the substrate or pigment. As a non-limiting example, addition of a water-soluble salt of a polyvalent metal, such as magnesium, calcium, aluminum, titanium, zinc or a zirconium salt (e.g., zirconium sulfate or chloride), or an alkaline salt, such as a sodium, potassium, lithium, ammonium, or an amine salt, can produce a chemical linkage. These metals typically are present in the form of a salt, such as a sulfate salt (e.g., aluminum sulfate, and the like). The reaction provides a surface-treatment agent chemically immobilized onto the surface of the cosmetic powder particle. In contrast, conventional coating a substrate or pigment with a surface-treatment agent involves absorbing the surface-treatment agent onto the surface of the substrate or pigment.

During treatment with a surface treatment agent, the surface of one or more cosmetic powders becomes modified. Including a cosmetically acceptable oil (a single oil or mixture of oils) during a treatment in which the cosmetic powder surface is modified invites oil at the same time as the particles become attached or linked to each other. Surface treatment agents and oil in combination function as a “glue” to attach or link particles, and other components optionally present, to each other. A mixture of two or more different cosmetic powders during such surface treatment results in forming composites, which are typically randomly and uniformly distributed onto the surface. Thus, oils, emulsifiers, etc., can be present in a mixture with one or more substrates and pigments when contacted with a surface treatment agent.

Following surface modification, a powder material can then be admixed or blended with another (e.g., second) powder material, such as a different pigment, or substrate or extender, or another cosmetically acceptable ingredient such as an oil, emulsifier, binder, etc. The second material may or may not have been treated with a surface treatment agent. Alternatively, two or more materials (e.g., different colored pigments), can be combined or mixed together prior to contact with a surface treatment agent, such as in an aqueous slurry, and then subsequently contacted with a polysaccharide surface treatment agent in order to simultaneously produce two or more surface modified or treated materials. Chemical immobilization of the polysaccharide and optionally other surface treatment agents on the powder materials can be facilitated by a water soluble compound having a lipophilic or hydrophilic moiety being absorbed onto the surface of the material, as set forth herein or known to the skilled artisan.

For cosmetic powders that are substrates, typical substrate sizes are about 1-200 microns in diameter, usually not less than 1 micron, for example, and may have a primary size of about 1-3 microns. Substrate particles typically are larger than pigment particles and have various shapes, for example, spherical, elliptical or “platy.” Substrates provide desirable texture and other characteristics such as smoothness, silkiness, round feel, moisture feel, optical benefits (soft focusing, hiding or concealing wrinkles or blemishes), etc.

Specific non-limiting examples of substrates include clay, mica (e.g., pearl colored mica, such as Timron Super Silver™, a mica coated with titanium dioxide produced by Rona/EMD Industries), talc, kaolin, sericite, silica (e.g., silica beads such as aluminum silicate, magnesium silicate and calcium sodium silicate, Beadyl Beads™, fumed silica), alumino-silicate minerals (zeolites), nylon (e.g., nylon beads or nylon powder), acrylates such as polymethyl methacrylate (PMMA or powder), metal powders (such as aluminum), ceramic powders (such as silicon nitride or boron nitride), cotton powder, wool powder, Microcrystalline cellulose, silk powder, cellulose and cellulose powder, urethane, polystyrene and polystyrene powder, polyolefin, polyethylene and polyethylene powder, polyamide, zirconium, aluminum oxide, zirconium oxide, starch, starch powder and starch derivatives such as aluminum starch octenylsuccinate, and calcium carbonate (chalk).

Substrates also may include “extenders.” An extender can function as a filler or bulking agent for powders and dispersions as set forth herein or known to the skilled artisan (e.g., pressed foundation, loose powder, blush, concealer, etc.). Extenders as a class typically have a size, shape or structure that is similar or identical to substrates as disclosed herein and understood by the skilled artisan. The term extender is typically used to refer to a substrate material that is added to a powder or dispersion after surface treatment or modification of cosmetic powder material.

Extenders include natural and synthetic substrates that may or may not have a color, shade, hue, chroma (saturation) or lightness that may vary in saturation and luminance. As with a substrate, an extender has a size typically greater than 1 micron (1 μm), for example about 1-30 microns, and can have various shapes, for example, spherical, elliptical or “platy.”

Non-limiting examples of extenders include talc, kaolin (clay), natural and synthetic micas including muscovite mica and sericite, titanated mica, cotton powder, starch, magnesium carbonate, calcium carbonate, aluminum silicate, magnesium silicate, calcium silicate, synthetic silicates, clay, bentonite, montmorillionite, calcite, chalk, bismuth oxychloride, boron nitride, fumed silica, silica beads, plastic beads such as acrylics, nylons such as Nylon 12, nylon beads, aluminum, calcium, or sodium silicate, and barium sulfate.

Cosmetic powder materials also may be comprised of pigments. As used herein, the term “pigment,” which includes “dyes” is a natural or synthetic material that has a certain color, shade, hue, chroma (saturation) or lightness. Pigments may be organic or inorganic in chemical nature. Pigments typically have a primary particle diameter not greater than about 3 microns. Pigments more typically are about one order of magnitude smaller in size than substrates, for example, about 0.01-1.0 microns in diameter. Other pigments, such as pearl pigments typically have a larger size, for example 10, 20, 30, 40, or 50-100 microns (μm). Thus, the cosmetic powder material, albeit a substrate, a pigment, or other powder, usually has an average particle size within the range of from about 0.01 to about 100 μm, or from about 0.05 to about 50 μm, or from about 0.1 to about 35 μm.

Non-limiting examples of inorganic pigments include white titanium dioxide pigments (e.g., rutile, anatase, and ultrafine TiO₂), zinc oxides (e.g., ultrafine ZnO), which can be of pigment grade and have a primary size of about 0.25 μm, or ultrafine grade, and have a primary size of less than about 0.1 μm. Other inorganic pigments include zirconium oxide, zirconium dioxides, iron oxides (including yellow, red, brown, green and black iron oxides), ultramarines (such as ultramarine blue, ultramarine violet, ultramarine pink, etc.), pearl pigments (e.g., mica, titanated mica, bismuth oxychloride, etc.), manganese violet, Prussian blue, chromium oxides, chromium hydroxides, and carbon black. Non-limiting examples of organic pigments include “lake” dyes, β-carotene, carmine, chlorophyll and the like.

The powder material may be an inorganic powder, such as an extender pigment, non-limiting examples of extender pigments include: mica, sericite, talc, kaolin, synthetic mica, muscovite, phlogopite, epidolite, biotite, calcium carbonate, magnesium carbonate, calcium phosphate, alumina, magnesium oxide, aluminum hydroxide, barium sulfate, magnesium sulfate, silicic acid, silicic anhydride, magnesium silicate, aluminum silicate, aluminum magnesium silicate, calcium silicate, barium silicate, strontium silicate, silicon carbide, magnesium aluminate, magnesium metasilicate aluminate, chlorohydroxyaluminum, clay, bentonite, zeolite, smectite, hydroxyapatite, ceramic powder, boron nitride and silica.

The powder material may be a special composite extender pigment such as, but not limited to Excel Mica, Excel Pearl and Powder La Vie sold by Miyoshi Kasei, Inc.; white pigments such as titanium dioxide, zinc oxide and cerium oxide; color pigments such as red iron oxide, yellow iron oxide, black iron oxide, chromium oxide, chromium hydroxide, Prussian blue, ultramarine, inorganic blue pigment, carbon black, titanium oxide, mango violet, cobalt violet, laked tar dye and laked natural dye; bright pigments such as bismuth oxychloride, mica titanium, fish scale guanine, a powder obtained by coating synthetic mica with titanium dioxide, a powder obtained by coating silica flakes with titanium dioxide as sold under a trade name “Metashine” by Nippon Sheet Glass Co., Ltd., a powder obtained by coating alumina flakes with tin oxide and titanium dioxide, a powder obtained by coating aluminum flakes with titanium dioxide, a powder obtained by coating copper flakes with silica as sold by Eckart, U.S.A., a powder obtained by coating bronze flakes with silica and a powder obtained by coating aluminum flakes with silica;

The powder material may be an organic powder, non-limiting examples of which include a wool powder, a polyamide powder, a polyester powder, a polyethylene powder, a polypropylene powder, a polystyrene powder, a polyurethane powder, a benzoguanamine powder, a tetrafluoroethylene powder, a polymethyl methacrylate powder, a cellulose powder, a silk powder, a silicone powder, a silicone rubber powder, a styrene acrylic copolymer, a divinylbenzene.styrene copolymer, synthetic resin powders such as a vinyl resin, a urea resin, a phenol resin, a fluoro resin, a silicon resin, an acrylic resin, a melamine resin, an epoxy resin and a polycarbonate resin, a fine crystalline fibrous powder, a starch powder, an acylated lysine powder, a long-chain alkyl phosphate metallic salt powder, or a metal soap powder.

Suitable powder materials include inorganic pigments such as, but not limited to titanium dioxides, zinc oxides, zirconium dioxides, iron oxides (including yellow, red, and black), ultramarines (such as ultramarine blue, ultramarine violet, etc.), and manganese violet. The powder material may be a mixture of any or all of the suitable powder materials.

The cosmetic powder whose surface has been modified with at least one polysaccharide or salts thereof usually will have an average treatment ratio of from about 0.1 to about 10% powder, by weight of polysaccharide (or from about 0.05 to about 20 parts of polysaccharide (including salt forms) by weight per 100 parts by weight of powder.

The powder also may be treated with from about 0.1 to about 10% powder, by weight of polysaccharide, or from about 2 to about 6% powder, by weight of polysaccharide, or from about 3 to about 5% powder, by weight of polysaccharide. The amount of polysaccharide surface treatment agent also may vary depending on the type of powder. For example, for ultrafine powder, such as silica having a larger surface area, more polysaccharide may be used, e.g., double or triple the amount used for powder having smaller surface area. Using the guidelines provided herein, persons having ordinary skill in the art will be capable of determining an appropriate amount of polysaccharide surface treatment agent to use, depending on the type of powder being treated.

A suitable polysaccharide used in the embodiments includes commercially available NOVEON, CP Kelco, LUBRISOL, KIMIKA and more. Polysaccharides include synthetic and natural polysaccharides. Suitable polysaccharides include, but are not limited to, alginic acid, alginic acid derivatives, starch, starch derivatives, cellulose, cellulose derivatives (CMC), chitin, chitosan and derivatives thereof, Xantham gum, as well as others. Useful polysaccharides in the embodiments described herein may be selected from one or more of the following formulae (as example):

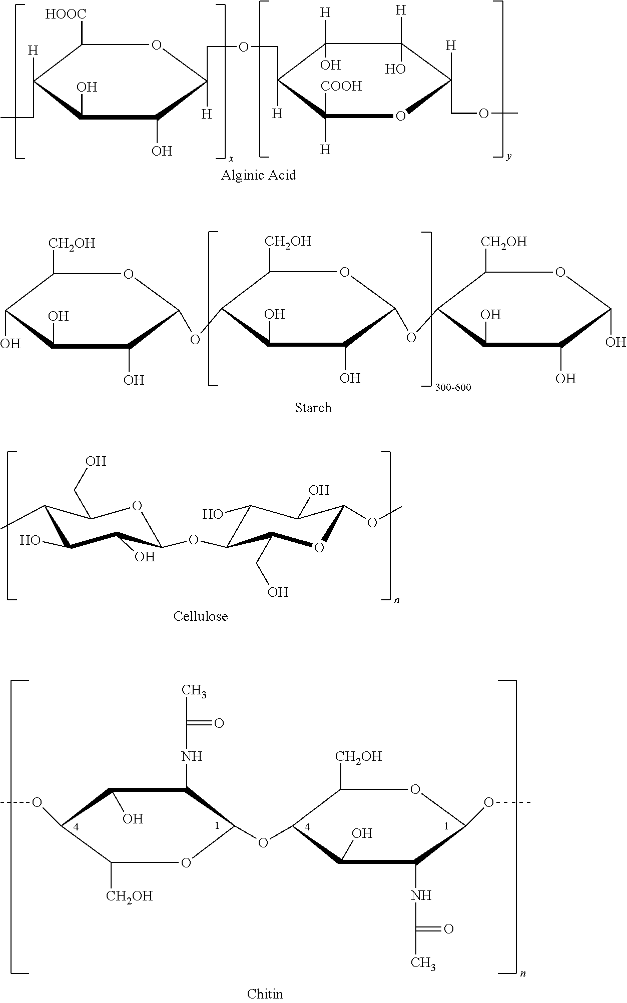

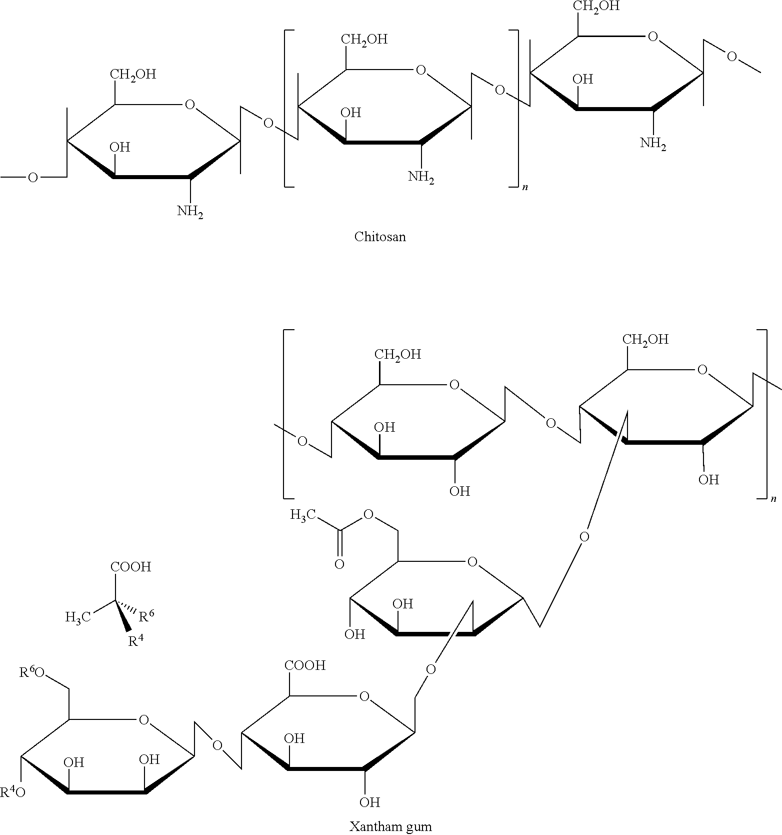

Polysaccharides or alkaline carboxylated polysaccharides also may be obtained microbiologically, for instance, by fermentation with Pseudomonas aeruginosa or mutants of Pseudomonas putida, Pseudomonas fluorescens or Pseudomonas mendocina.

Polysaccharides useful in the embodiments typically have both hydroxyl and carboxyl groups in the structure. As such, there are reactive sites to react to chemically immobilize onto the surface of the cosmetic powder material. Polysaccharides can be used as is or can be present in the form of one or more salts thereof, which can be converted into the polysaccharide and used as starting material for the purposes of the embodiments.

The surface treated cosmetic powder of the embodiments therefore can be comprised of at least one cosmetic powder having a modified surface in which at least one polysaccharide, or salts thereof, is chemically immobilized on the surface of the powder material. The treated powder material may be used as is in a cosmetic composition, or it may be further treated with one or more additional surface treatment agents. Specific non-limiting classes of surface treatment agents include surface active agents, which include surfactants, detergents, wetting agents and emulsifiers. Surface-active agents may be nonionic, anionic, cationic, amphoterics, hydrophobic or hydrophilic.

Surface-treatment agents typically have one or more reactive groups, such as a hydrophilic moiety (e.g., a carboxyl group, a phosphorous group, a sulfur group, a silanol group or a silane group) or hydrophobic moiety (e.g., a hydrocarbon, a dialkyl(CH₃-, C₂H₅-) polysiloxane, perfluoroalkyl, etc.) in their structure. Surface-treatment agents may or may not contain one or more hydroxyl groups or alkylene oxide moieties, such as ethylene oxide or propylene oxide. Those having hydroxy groups in their structure and hydrophilic characteristics can be delivered after completing the reaction onto the surface.

Non-limiting examples of surface treatment agents include acyl collagens, ether carboxylic acids, lactic acid, gluconic acid, galacturonic acid, glucarolactone, gallic acid, glucoheptanoic acid, amino acids (such as thereonine and serine) and their salts, acyl amino acids (such as acylglutamates, acylsarcosinates, acylglycinates, and acylalaninates), fatty acids and their salts, and glycerol phosphate esters (such as lecithin). Additional non-limiting examples of surface-treatment agents include methicone, dimethicone and polyethylenes with free carboxylic acids.

Examples of anionic surface active agents (surfactants) include soaps (fatty acids/alkyl carboxylic acids salt), hydroxy fatty acids, alkyl sulfate, alkyl ether phosphate, polyoxyalkylene alkyl ether sulfate, polyoxyalkylene alkyl ether carboxylate, alkylether phosphate, acyl N-methyl taurate, N-acylamino acid salts (glutamate, sarcosinate, lalaninate, glycinate, B-alaninate), acyl peptides (acyl collagen, acyl silk protein), sodium cocoate, stearic acid, iso-stearic acid, potassium palmitate, sodium laurate, 12-hydroxystearic acid, sodium lauryl sulfate, sodium myristyl phosphate, sodium myristoyl sarcosinate, sodium polyoxyethylene lauryl sulfate, polyoxyethylene myristyl carboxylate, potassium myristate, zinc gluconate, isostearyl sebacic acid, sodium myristoyl taurate, disodium stearoyl glutamate, disodium cocoyl glutamate, arginine lauryl glycinate, sodium dilauramidoglutamide lysine.

Suitable surface treatment agents may include one or more of the surface treatment agents disclosed in, for example, U.S. Pat. No. 6,887,494, U.S. Patent Application Publication Nos. 2008/0299158, 2011/0318286, the disclosures of which are incorporated by reference herein in their entireties.

The cosmetic powders may be used in cosmetic compositions that comprise the surface treated powder and a cosmetically acceptable vehicle. In an embodiment, the surface treated powder is present in an amount within the range of from about 0.1% to about 50% by weight of the composition, or from about 0.5% to about 30%, or from about 1% to about 20% by weight, based on the weight of the composition.

Depending on the formulation (e.g., liquid formulation, powder formulation, skin lotion, body soap, Shampoo, Conditioner, Hair Styling, etc.), the amount of the powder can vary widely. For example, for a powder formulation, such as makeup foundation or the like, the amount of the polysaccharide treated powder can be used in an amount of from about 5 to about 50% by weight, or from about 15 to about 40% by weight, or from about 25 to about 35% by weight, or at about 30% by weight. For a skin lotion formulation, the amount of the polysaccharide treated powder can be used in an amount of from about 0.1 to about 15% by weight, or from about 1 to about 10% by weight, or from about 2 to about 7% by weight, or at about 5% by weight. For a body soap formulation, the amount of the polysaccharide treated powder can be used in an amount of from about 2 to about 40% by weight, or from about 5 to about 20% by weight, or from about 7 to about 15% by weight, or at about 10% by weight

The cosmetic compositions useful in the embodiments described herein also may contain other conventional components useful in various cosmetic compositions. Any cosmetically acceptable vehicle may be used together with the polysaccharide treated powder material. Such vehicles may include, for example, water, glycerin, dimethicone, beeswax, glyceryl stearate, and the like. Other ingredients normally used in cosmetics also may be present, when desired. For example, inorganic powders such as talc, kaolin, sericite, muscovite, phlogopite, red mica, biotite, synthetic mica, lithia mica, vermiculite, magnesium carbonate, calcium carbonate, diatomite, magnesium silicate, calcium silicate, aluminum silicate, barium silicate, barium sulfate, strontium silicate, wolframic acid metal salt, or silica, hydroxyapatite, zeolite, boron nitride, ceramic powder, organic powders such as nylon powder, polyethylene powder, polystyrene powder, benzoguanamine powder, polyfluoridation ethylene powder, di-styrene benzene polymer powder, epoxy powder, acrylic powder, silicone powder, microcrystalline cellulose, inorganic white pigments such as titanium dioxide and zinc oxide, inorganic red system pigments such as iron oxide (red iron oxide) and titanic acid irons, inorganic brown system pigments such as γ-iron oxides, inorganic yellow system pigments such as yellow soil and yellow iron oxides, inorganic black color system pigments such as tetravalent acid iron oxide, carbon black, inorganic violet system pigments such as mango violet, cobalt violet, inorganic green system pigments such as chromium oxide, chromium hydroxide, and titanic acid cobalt, inorganic blue system pigments such as ultramarine blue, and prussian blue, pearl pigments such as titanium dioxide covered mica, titanium dioxide covered bismuth oxychloride, bismuth oxychloride, titanium dioxide covered talc, fish scale foil, colored titanium dioxide covered mica, metal powder pigment such as aluminum powder, copper powder, colored composite pigments such as iron-doped zinc oxide and iron-doped titanium dioxide.

Other pigments may be used, such as red No. 201, red No. 202, red No. 204, red No. 205, red No. 220, red No. 226, red No. 228, red No. 405, orange-colored No. 203, orange-colored No. 204, yellow No. 205, yellow No. 401 and blue No. 404, organic chlorophyll pigment such as FD&C Red No. 3, red No. 104, red No. 106, red No. 227, red No. 230, red No. 401, red No. 505, orange-colored No. 205, FD&C Yellow No. 4, yellow No. 5, yellow No. 202, yellow No. 203, orange-colored No. 3 and zirconium, barium, or aluminum lake of blue No. 1, natural colorants such as β-carotene, hydrocarbon oils such as squalane, mineral oil, petroleum jelly, micro crystalline wax, ozokerite, ceresin, myristic acid, palmitic acid, stearic acid, oleic acid, iso-stearic acid, cetyl alcohol, hexadecyl alcohol, oleyl alcohol, cetyl 2-ethylhexanoate, 2-ethylhexyl palmitate, 2-octyldodecyl myristate, neo-pentylglycol di-2-ethylhexanoate, glyceryl tri-2-ethylhexanoate, 2-octyldocyl oleate, isopropyl myristate, glyceryl triisostearate, caprylic/capric triglyceride, olive oil, avocado oil, yellow bees wax, myristyl myristate, mink oil, lanolin oil, silicone oil, higher fatty acid oil, ester oils of fatty acids, higher alcohol, oil components of wax groups, cyclopentasiloxanes, dimethicones, trimethylsiloxysilicates, and organic solvents such as acetone, toluene, butyl acetate, and ester acetate can be used in various amounts.

Resins such as alkyd resin, urea-formaldehyde resin, Nylon-12, plasticizers such as camphor, acetyl tributyl citric acid, ultraviolet absorbing agents, antioxidants, antiseptics, emulsifiers, surfactants, stabilizers, defoamers, moisturizing agents, perfumes, water, alcohol, and thickeners can also be used. Non-limiting examples of emulsifiers include cetyl dimethicone copolyol, polygyceryl-4 isosteatrate, glyceryl stearate, PEG-100 stearate, cetyl alcohol, dicetyl phosphate, and ceteth-10 phosphate isostearic acid.

Surfactants typically include nonionic forms. Non-limiting examples of nonionic surfactants include polyoxyalkylene (PEG or/and PPG) type nonionic emulsifiers having structures:

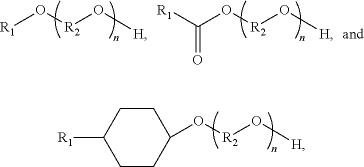

wherein R₁ is selected from the group consisting of alkyl, alkylamide, alkenyl, alkynyl, alkoxy, aryl, cycloalkyl, and arylalkyl group, each of which may be substituted by one or more hydroxy group, and may further be substituted by one or more alkoxyl, carboxyl, or oxo group. R₁ has a carbon number of C₈ to about C₂₄; R₂ is selected from the group consisting of —C₂H₄—, —C₃H₆—, and —C₄H₈—.

The powder surface treated with at least one polysaccharide can be prepared by preparing an aqueous polysaccharide solution by mixing at least water and at least one polysaccharide or a salt thereof, and adding a cosmetic powder to the solution with high speed to homogenize the mixture to a homogenized powder mixture that is uniformly dispersed. The homogenized powder mixture then can be contacted with a neutralizing agent (e.g., Al₂(SO₄)₃), to neutralize the homogenized powder mixture, and to chemically immobilize the at least one polysaccharide to the surface of the powder. The method may further include filtering and drying the powder to produce a powder having its surface modified with polysaccharide.

In certain embodiments, anywhere from about 1 to about 10 g, or from about 2 to about 8, or from about 3 to about 5 g of polysaccharide, or a salt thereof, is added to from about 90 to 99, or from about 92 to about 98, or from about 95 to about 97 g of water. The embodiments therefore include the use of anywhere from about 1 to 10 parts by weight of polysaccharide, or salt thereof, or from about 2 to about 8, or from about 3 to about 5, or about 3 parts by weight polysaccharide, or a salt thereof, added to from about 90 to 99, or from about 92 to about 98, or from about 95 to about 97 parts by weight of water. The polysaccharide, or salt thereof, can be added to the water at a temperature of from about 25 to about 75° C., or from about 40 to about 60° C., or at about 50° C., and mixed in a disperser for a period of time sufficient to homogenize the mixture. Any dispersing and/or mixing apparatus can be used. A suitable disperser may include a ROBOMIX® disperser, commercially available from Primix Corporation, Osaka, Japan. The mixture can be mixed for anywhere from about 10 minutes to an hour, or from about 15 minutes to 45 minutes, or from about 18 minutes to 30 minutes, or for about 20 minutes, until the mixture is adequately homogenized.

A cosmetic powder then can be added to the homogenized mixture, with stirring. Any of the cosmetic powders described herein can be used, including pigments, substrates, and extenders. The amount of powder added will vary, depending on the amount of water used, and generally is added in an amount of from about 50 to about 150% by weight, based on the weight of the water, or from about 75% to about 125%, or from about 90% to about 110%, or from about 98% to about 105%, or about the same amount of water. In one embodiment, from about 50 to about 150 grams of powder are added, or from about 75 to about 125 g, or from about 90 to about 110 g, or from about 98 to about 105 g, or about 97 g. of powder are added. The powder may have a particle size anywhere within the range of from about 0.1 to about 0.3 μm, or about 0.25 μm, and can be mixed in the dispersing and/or mixing apparatus for a period of time sufficient to adequately disperse the cosmetic powder. The powder can be mixed for a period of time within the range of from about 5 to about 60 minutes, or from about 10 to about 40 minutes, or from about 15 to about 25 minutes, until adequately dispersed.

A suitable neutralizing agent then may be added to the powder-containing mixture to bring the pH of the mixture to a value within the range of from about 2 to about 10, or from about 3 to about 8, or from about 4 to about 7, or about 4.0. Any neutralizing agent can be used in the embodiments, and a suitable neutralizing agent is aluminum sulfate. The neutralizing agent can be metered into the mixture until the pH reaches the desired value. Once the final pH is reached, the product then can be recovered from the mixture using any suitable mechanism, including filtration, and then drying. The powder treated with polysaccharide can be dried at a temperature of between about 75 to about 200° C., or from about 90 to about 150° C., or at about 105° C., for a period of time sufficient to dry the powder. The powder treated with polysaccharide may be subjected to drying for a period of from about 5 to about 35 hours, or from about 10 to about 20 hours, or from about 15 to about 17 hours, or about 16 hours, to produce, in the embodiments disclosed above, about 100 g of powder treated with polysaccharide (97 g of powder were added to a mixture containing about 3 g of polysaccharide, or a salt thereof, to produce about 100 g of powder treated with polysaccharide).

The powder treated with polysaccharide can be used in a cosmetic composition that contains conventional cosmetic additives. For example, the composition may include up to about 25 wt % of a non-volatile oil. The non-volatile oil may be comprised of an organic, UV-active material that functions as a UV-protective agent (a “sun block”). Preferably, two or more organic, UV-actives are used to provide a wide spectrum of protection in the UV region. For example, a combination of at least one UV protecting agent that mainly provides protection against UVA light, and at least one UV protecting agent that mainly provides protection against UVB light, may be used.

A wide variety of conventional UV protecting agents are suitable for use herein. Non-limiting exemplary organic, UV-actives include: 2-ethylhexyl-p-methoxycinnamate (commercially available as PARSOL MCX), butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, 2-ethylhexyl N,N-dimethyl-p-aminobenzoate, p-aminobenzoic acid, 2-phenylbenzimidazole-5-sulfonic acid, octocrylene (Parsol 340, DSM), oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, Eusolex™ 6300, avobenzone (Parsol 1789, DSM), avobenzone, PABA, octyldimethyl-PABA, Phenylbenzimidazole sulfonic acid, Cinoxate, Dioxybenzone (Benzophenone-8), Oxybenzone (Benzophenone-3), Homosalate, Menthyl anthranilate, Octisalate, Sulisobenzone, Trolamine salicylate, Terephthalylidene Dicamphor Sulfonic Acid, 4-Methylbenzylidene camphor, Methylene Bis-Benzotriazolyl Tetramethylbutylphenol, Bis-ethylhexyloxyphenol methoxyphenol triazine, bisimidazylate, Drometrizole Trisiloxane, Octyl triazone, Diethylamino Hydroxybenzoyl Hexyl Benzoate, Iscotrizinol, Polysilicone-15, Amiloxate, Ethylhexyl Dimethoxybenzylidene Dioxoimidazolidine Propionate, and mixtures thereof.

In addition to a UV-active, the non-volatile oil may comprise an ancillary oil which may be a solvent for one or more of the UV-active oils. The ancillary oil may provide desirable cosmetic properties such as emolliency and a good “skin feel.” A preferred, but non-limiting ancillary oil is isopropyl myristate.

Non-volatile cosmetic emollient oils having a relatively high boiling point and function as a skin feel modifiers include, but are not hydrocarbons, fatty alcohols, fatty acids, non-volatile silicone oils, and esters such as glycerides and glycol esters.

Suitable ancillary oils include, but are not limited to isotridecyl isononanoate, isostearyl isostearate, isocetyl isosteatrate, isopropyl isostearate, isodecyl isonoanoate, cetyl octanoate, isononyl isononanoate, isocetyl myristate, isotridecyl myristate, isopropyl myristate, isostearyl palmitate, isocetyl palmitate, isodecyl palmitate, isopropyl palmitate, octyl palmitate, caprylic/capric acid triglyceride, glyceryl tri-2-ethylhexanoate, neopentyl glycol di(2-ethyl hexanoate), diisopropyl dimerate, tocopherol, tocopherol acetate, avocado oil, camellia oil, turtle oil, macadamia nut oil, corn oil, mink oil, olive oil, rapeseed oil, egg yolk oil, sesame oil, persic oil, wheat germ oil, pasanqua oil, castor oil, linseed oil, safflower oil, cotton seed oil, perillic oil, soybean oil, peanut oil, tea seed oil, kaya oil, rice bran oil, china paulownia oil, Japanese paulownia oil, jojoba oil, rice germ oil, glycerol trioctanate, glycerol triisopalmiatate, trimethylolpropane triisostearate, glycerol tri-2-ethylhexanoate, pentaerythritol tetra-2-ethylhexanoate, lanolin, liquid lanolin, liquid paraffin, squalane, vaseline, and mixtures thereof. Commercially available oils include, for example, tridecyl isononanoate with tradename Crodamol TN available from Croda, Hexalan available from Nisshin Seiyu, and tocopherol acetates available from Eisai.

Non-volatile cosmetic emollients may include waxes such as, but not limited to paraffin wax, microcrystalline wax, ozokerite wax, ceresin wax, carnauba wax, candelilla wax, and eicosanyl behenate.

Non-volatile silicon oils may be used including, but not limited to polymethylphenylsiloxane, polydiphenylsiloxane, polydiethylsiloxane, polydimethylsiloxane (dimethicone). For purposes of the present disclosure, a non-volatile silicon oil is defined as one that has a kinematic viscosity greater than 10 centi Stokes (cSt).

Suitable ancillary oils include polyalkyl or polyaryl siloxanes as disclosed in U.S. Pat. No. 6,936,241, the disclosure of which is incorporated by reference herein in its entirety.

Suitable ancillary oils useful herein include the various grades of mineral oils. Mineral oils are liquid mixtures of hydrocarbons that are obtained from petroleum. Specific examples of suitable hydrocarbons include paraffin oil, mineral oil, dodecane, isododecane, hexadecane, isohexadecane, eicosene, isoeicosene, tridecane, tetradecane, polybutene, polyisobutene, and mixtures thereof.

The non-volatile oil may not comprise a “volatile” silicone oil. A specifically excluded volatile silicone oil is decamethylcyclopentanasilaxane, commonly known as “D5.”

The foregoing description of the embodiments illustrates and describes the preferred embodiments but, as mentioned above, it is to be understood that the embodiments are capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing them and to enable others skilled in the art to utilize the embodiments in such, or other, embodiments and with the various modifications required by the particular applications or uses. Accordingly, the description is not intended to limit the embodiments to the form disclosed herein. Also, it is intended that the appended claims be construed to include alternative embodiments.

Throughout this application, various references including publications, patents, and pre-grant patent application publications are referred to. Disclosures of these publications in their entireties are hereby incorporated by reference into this application to more fully describe the state of the art to which the embodiments pertain. It is specifically not admitted that any such reference constitutes prior art against the present application or against any claims thereof All publications, patents, and pre-grant patent application publications cited in this specification are herein incorporated by reference, and for any and all purposes, as if each individual publication or patent application were specifically and individually indicated to be incorporated by reference. In the case of inconsistencies the present disclosure will prevail.

The embodiments now will be explained in greater detail with reference to the following non-limiting examples.

### EXAMPLES

### Example 1

### Preparation of Polysaccharide Treated Powder

The present example discloses how to treat a cosmetic powder with polysaccharide. While the example describes treating titanium dioxide with polysaccharide, the same method was used to treat other powders used in the examples described herein.

Approximately 97 grams of water were mixed together with about 3 grams of polysaccharide, at about 50° C. using a ROBOMIX® disperser, commercially available from Primix Corporation, Osaka, Japan, for a period of about 20 minutes. The polysaccharide included, for example, one or more of alginic acid, alginic acid derivatives, starch, starch derivatives, cellulose, cellulose derivatives (CMC), chitin, chitosan, Xantham gum, and derivatives thereof. To this homogenous mixture then were added about 97 grams of titanium dioxide powder commercially available from Ishihara Corp., San Francisco, Calif., and mixed until homogenized. To this homogenized powder-containing solution then were added stepwise by metering, aluminum sulfate, until the pH of the mixture reached a value of 4.0. The product obtained then was separated from the mixture and dried at a temperature of about 105° C. for 16 hours to produce about 100 grams of titanium dioxide powder treated with polysaccharide.

### Comparative Example

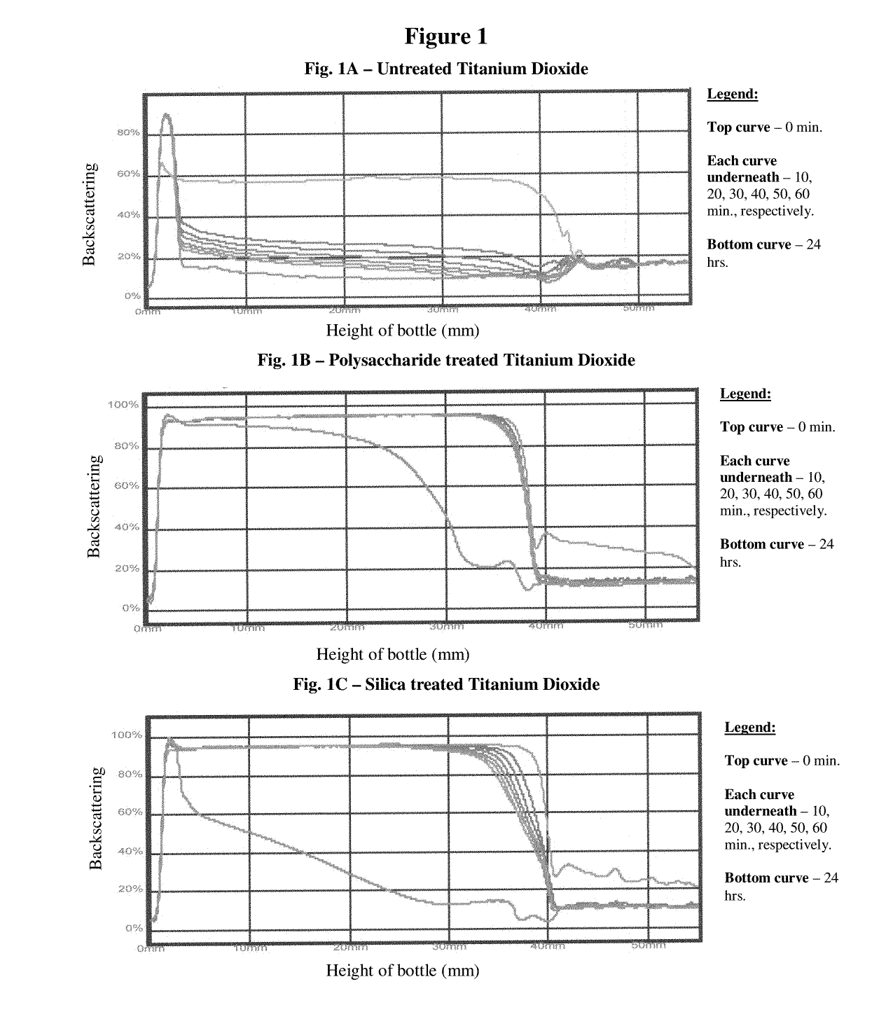

The titanium dioxide treated with polysaccharide prepared in accordance with Example 1 was then subjected to a dispersibility test using a Turbiscan™ LAB, commercially available from Formulaction Inc., L'Union, France, with measurements taken at 0, 10, 20, 30, 40, 50, 60 minutes, and at 24 hours. Uncoated titanium dioxide, and titanium dioxide coated with silica using conventional coating techniques, also was subjected to the same dispersibility test for comparison purposes. The results are shown in FIG. 1.

As shown in FIG. 1, the untreated titanium dioxide in FIG. 1A and the conventionally coated titanium dioxide (coated with silica) in FIG. 1C, both had inferior dispersibility when compared to the titanium dioxide treated with the polysaccharide of Example 1, as shown in FIG. 1B. Generally, powder naturally settles down little by little after mixing into liquid, and if the powder does not have good dispersibility, it tends to agglomerate in the liquid and sedimentation velocity increases. The dispersibility testing equipment measures transmitted light through the bottle containing powder dispersion. If the powder has good dispersibility and the powder does not agglomerate and stays suspended longer, transmitted light is detected only on the upper part of the bottle. If the powder does not have great dispersibility, it agglomerates, sedimentation velocity is fast and transmitted light is detected not only in the upper part of bottle but also in the middle and bottom parts of the bottle.

Powder settling was measured at 10, 20, 30, 40, 50 and 60 minutes and 24 hours. The horizontal axis shows the height from the bottom to the surface of the water (left side is bottom). As the line moves from right to left it means that the powder settles down. If the line does not change position, it means that the powder does not settle down and indicates good dispersibility.

The untreated powder line in FIG. 1A moves to left very quickly (first line is Orange, next 10 min line is blue). The silica coated powder line (FIG. 1C) also moves to the left easily, but the polysaccharide treated powder line in FIG. 1B stays relatively constant. These test results show polysaccharide treated powder has significantly better dispersibility than untreated and conventional silica coated powders.

A powder that disperses well, that does not agglomerate, and that has anti-caking properties is beneficial for the manufacture and stability of cosmetic formulations. Untreated powders often exhibit poor dispersibility and product stability, resulting in aggregates, agglomerates and flocculates due to the nature of powder's physical properties including particle size, surface activity, charge, polarity and specific gravity etc.

### Example 2

### Preparation of Powder Foundation

Mica was treated with the polysaccharide Xantham gum in accordance with the procedure described in Example 1. The mica treated with polysaccharide then was formulated into a powder foundation formulation as shown in Table 1 below. The same powder foundation also was prepared using mica that was not treated with polysaccharide, and compared by measuring the feeling, dispersibility, and stability of the respective compositions. The un-treated mica is listed as comparative example 2 in Table 1 below.

Sensory Feeling (Smoothness): Human questionnaire

5: excellent, 4: Good, 3: average, 2: fair, 1: poor)

Dispersibility: keep the sample in the oven 50° C. and measure. Dispersibility using Turbiscan™ LAB as previously described.

5: 1 month later less than 50% settle down, 4: 1 month later more than 50% settle down, 3: 3 week later settle down, 2: 2 week later settle down, 1:, 1 week later settle down

Wash-Ability: Wash Homogenizer head by flow water 1 min.

5: Completely washed away, 4: 1-2 particle found on one/limited area, 3: 1-2 particle found on several areas, 2: uncountable particle found on one area, 1:, uncountable particle on several areas.

Stability: keep the sample in the oven at 50 degree C.

5: 1 month no change, 4: 3 week no change 3: 2 week no change, 2: 1 week no change, 1:, 1 day change

As shown in the table above, the powder foundation formulation that contained the polysaccharide -treated cosmetic powder (mica) provided superior feeling and stability when compared to a powder foundation formulation that contained un-treated mica.

### Example 3

### Preparation of Skin Toner Formulation

Iron oxide was treated with the polysaccharide alginic acid in accordance with the procedure described in Example 1. The iron oxide treated with polysaccharide was then formulated into a skin toner formulation as shown in Table 2 below. The same skin toner formulation was also prepared using iron oxides that were not treated with polysaccharide, and then compared by measuring the feeling, dispersibility, and stability of the respective compositions. The iron oxide that was not treated with polysaccharide is listed as comparative example 3 in Table 2.

As shown in the table above, the skin toner formulation prepared in accordance with the present embodiments, which included polysaccharide -treated cosmetic powders, had significantly improved dispersibility and stability when compared to skin toner formulations prepared using un-treated cosmetic powders.

### Example 4

### Preparation of Skin Lotion

Talc was treated with the polysaccharide cellulose in accordance with the procedure described in Example 1. The talc treated with polysaccharide then was formulated into a skin lotion formulation as shown in Table 3 below. The same skin lotion foundation also was prepared using talc that was not treated with polysaccharide, and compared by measuring the feeling, dispersibility, and stability of the respective compositions. The talc that was not treated with polysaccharide is listed as comparative example 4 in Table 3.

As shown in the table above, the skin lotion formulation that contained the polysaccharide -treated cosmetic powder (talc) provided significantly improved dispersibility and stability when compared to a powder foundation formulation that contained un-treated talc.

### Example 5

### Preparation of Body Soap Formulation

Kaolin was treated with the polysaccharide starch in accordance with the procedure described in Example 1. The kaolin treated with polysaccharide then was formulated into a body soap formulation as shown in Table 4 below. The same body soap formulation also was prepared using kaolin that was not treated with polysaccharide , and compared by measuring the feeling, dispersibility, and stability of the respective compositions. The kaolin that was not treated with polysaccharide is listed as comparative example 5 in Table 4.

As shown in the table above, the body soap formulation that contained the Polysaccharide-treated cosmetic powder (kaolin) provided superior feeling, dispersibility, and stability when compared to a body soap formulation that contained un-treated kaolin.

### Example 6

### Easy Wash Ability

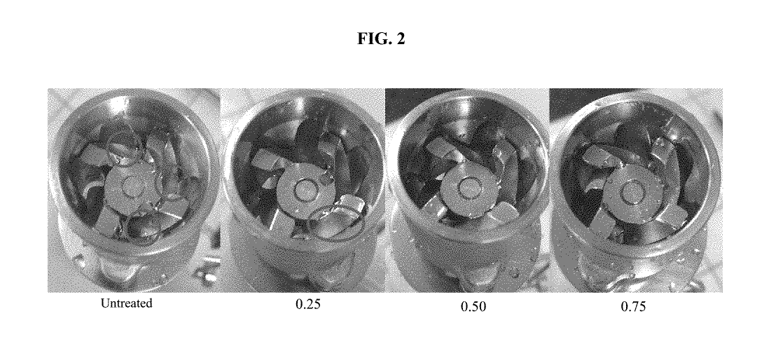

Pearl lucent pigment was treated with polysaccharide in accordance with the procedure described in Example 1. The pearl treated pigment then was subjected to a wash-ability test using a Human test and Homogenizer test, commercially available from PRIMIX, Japan, with measurements taken at 3 hours(Human test)/10 minutes(Homogenizer test). Uncoated and several pigments treated with varying amounts of polysaccharide were also subjected to the same easy wash-ability test for comparison purposes. The results are shown in Table 5 and FIG. 2.

### Washability Test Method

Table5; Apply the un-treated/treated pigment with polysaccharide on human skin and wash by following water and observe the condition.

TABLE 5

Washing Time

| | 5 min | 10 min | 15 min | 20 min |
|---|---|---|---|---|
| Treated | 4 | 4 | 5 | 5 |
| Untreated | 1 | 2 | 2 | 2 |

5: Completely washed out,
4: Most pigment washed out,
3: Half of pigment wash out/remain,
2: Most pigment remain,
1: Completely pigment remain.

5: Completely washed out, 4: Most pigment washed out, 3: Half of pigment wash out/remain, 2: Most pigment remain, 1: Completely pigment remain.
FIG. 2; Prepare O/W emulsion which contains the pigment treated with polysaccharide by Homogenizer. Wash homogenizer with flowing water and observe the homogenizer head.

As shown in FIG. 2, the untreated pigment (the left-most homogenizer in the figure) was not effectively removed during the test washing procedure described immediately above. FIG. 2 also shows the washability of powders treated with varying amounts of polysaccharide.

The invention has been described with reference to particularly preferred embodiments. Those having ordinary skill in the art will readily appreciate that various modifications may be made to the invention without departing from the spirit and scope thereof.

1. A powder comprising at least one cosmetic powder in which the surface of at least one cosmetic powder is chemically modified with at least one polysaccharide, and salt forms and mixtures thereof, wherein the at least one polysaccharide is chemically immobilized on the surface of at least one powder, and wherein the powder exhibits improved washability, when compared to untreated powder, when the at least one cosmetic powder is chemically modified with as little as 0.25% of polysaccharide.

2. The powder of claim 1, wherein the at least one cosmetic powder is selected from the group consisting of inorganic pigment powders, organic pigment powders, powder substrates, extenders, extender pigments, and mixtures thereof.

3. The powder of claim 2, wherein the at least one cosmetic powder is selected from the group consisting of mica, kaolin, talc, titanium dioxide, iron oxide, pearlescent pigment and mixtures thereof.

4. The powder of claim 3, which has a superior dispersibility in an aqueous phase and superior washability, when compared to the same powder comprising at least one cosmetic powder that was not treated with at least one polysaccharide.

5. The powder of claim 1, wherein the at least one polysaccharide is selected from the group consisting of alginic acid, alginic acid derivatives, starch, starch derivatives, cellulose, cellulose derivatives, chitin, chitosan, Xantham gum, and mixtures and salt forms thereof.

6. The powder of claim 1, wherein the at least one polysaccharide, or salt form thereof, is present in an amount from about 0.05 to about 20 parts by weight, based on 100 parts by weight of the at least one cosmetic powder.

7. The powder of claim 6, wherein the at least one polysaccharide, or salt form thereof, is present in an amount from about 0.1 to about 10 parts by weight, based on 100 parts by weight of the at least one cosmetic powder.

8. The powder of claim 7, wherein the at least one polysaccharide, or salt form thereof, is present in an amount from about 0.1 to about 6 parts by weight, based on 100 parts by weight of the at least one cosmetic powder.

9. A method of making a surface-modified cosmetic powder comprising:
(a) preparing an aqueous solution of at least one polysaccharide, and salt forms and mixtures thereof;
(b) adding to the aqueous solution at least one cosmetic powder with agitation to uniformly disperse the powder in the aqueous mixture; and
(c) adding a metal-containing salt to neutralize the aqueous mixture and immobilize the at least one polysaccharide on the surface of the at least one cosmetic powder.

10. The method of claim 9, wherein the metal-containing salt is aluminum sulfate.

11. The method of claim 9, wherein the metal-containing salt is added step-wise until the pH of the solution is within the range from about 3 to about 8.

12. The method of claim 11, wherein the metal-containing salt is added step-wise until the pH of the solution is about 4.0.

13. The method of claim 9, wherein the powder is selected from the group consisting of inorganic pigment powders, organic pigment powders, powder substrates, extenders, extender pigments, and mixtures thereof.

14. The method of claim 13, wherein the powder is selected from the group consisting of mica, kaolin, talc, titanium dioxide, iron oxide, pearlescent pigment and mixtures thereof.

15. The method of claim 9, wherein the at least one polysaccharide, or salt form thereof, is present in an amount from about 0.05 to about 20 parts by weight, based on 100 parts by weight of the at least one cosmetic powder.

16. The method of claim 15, wherein the at least one polysaccharide, or salt form thereof, is present in an amount from about 2 to about 6 parts by weight, based on 100 parts by weight of the at least one cosmetic powder.

17. A cosmetic composition comprising:
(a) at least one cosmetic powder in which the surface of at least one cosmetic powder is chemically modified with at least one polysaccharide, and salt forms and mixtures thereof, wherein the polysaccharide is chemically immobilized on the surface of at least one powder; and
(b) a cosmetically acceptable carrier.

18. The cosmetic composition of claim 17, wherein the composition is in the form selected from the group consisting of a powder foundation, liquid foundation, point makeup, lip, mascara, eyeliner, skin care products which are skin cream, hair care products which are shampoo, conditioner, treatment and hair styling products, hair color products, cleansing products which are a body soap, hand soap and facial cleanser.

19. The cosmetic composition of claim 17, wherein the at least one cosmetic powder is selected from the group consisting of inorganic pigment powders, organic pigment powders, powder substrates, extenders and extender pigments.
